(19) United States
(12) Patent Application Publication
(10) Pub. No.: US 2004/0029288 A1
(43) Pub. Date: Feb. 12, 2004

(54) Nonlinear gold nanocluster chemical vapor sensor

(76) Inventors: Arthur Snow, Alexandria, VA (US); Mario Ancona, Alexandria, VA (US); Edward Foos, Alexandria, VA (US)

Correspondence Address: NAVAL RESEARCH LABORATORY, ASSOCIATE COUNSEL (PATENTS), CODE 1008.2, 4555 OVERLOOK AVENUE, S.W., WASHINGTON, DC, 20375-5320, US

(21) Appl. No.: 10/145,617

(22) Filed: Aug. 9, 2002

(51) Int. Cl. G01N 33/00; G01N 27/02

(52) U.S. Cl. 436/149; 436/84; 436/80; 436/111; 436/119; 436/183; 422/82.01; 422/82.02

(57) ABSTRACT

The present invention is a chemiresistor for qualitative and quantitative analysis of chemical species that consists of a very thin film of particles, nanoclusters, deposited on an insulating substrate and contacted by electrodes. The particles have a metallic core, preferably spheroidal, that is less than 5 nm in diameter and surrounded by an monolayer ligand shell ranging in thickness from 0.4 nm to 2 nm. The Coulomb blockade effects upon which the invention operates result in nonlinear current-voltage characteristics which dramatically improves sensitivity with much lower power dissipation. One property of the ligand shell is that its chemical composition can be chosen to be especially receptive to a particular chemical vapor.

### BACKGROUND OF THE INVENTION

1. Field of the Invention

This invention relates to chemical sensing devices and, more specifically, to the qualitative and quantitative analysis of a chemical species in a target environment wherein the properties of certain nanoclusters interact with the chemical species such that they can be monitored as an indication of whether the species is present and in what amount.

2. Description of the Background Art

There are a number of well-known approaches for determining the presence and amount of a chemical species in a target environment by exposing a substance capable of interacting with the species to such environment and monitoring a change in a property of that substance due to such interaction as an indication of whether or in what amount the species is present.

One such approach has been the exposure to the environment of a species-interactive substance applied to a piezoelectric substrate. The substance is affected such that, if any of the species present, a preselected property of the substance is changed. A surface acoustic wave is induced in the piezoelectric material. Any change of property in the substance results in an attenuation of the surface acoustic wave, which can be monitored as an indication of whether or in what amount the species is present. For instance, see U.S. Pat. Nos. 4,312,228 and 4,759,210.

Another approach has been the provision of a capacitive device for detecting the presence or measuring the concentration of an analyte in a fluid medium. A plurality of interdigitated fingers formed from metallic conductors are placed upon an insulating substrate. The substrate may be made from an insulating material such as glass and the fingers may be made of copper and gold; the fingers are covered with an insulating passivation layer. This approach involves biospecific binding between a biochemical binding system and the analyte to change the dielectric properties of the sensor. See U.S. Pat. No. 4,822,566.

Yet another approach has been the provision of a chemical sensor comprising a thin film of dithiolene transition metal complexes applied to a chemiresistor device. The film is deposited upon an interdigitated electrode on a substrate. The film changes conductivity when exposed to a chemical gas or vapor of analytical interest. The interdigitated electrodes may be gold and the substrate is an insulating material such as quartz. A power supply and current measuring device are included. See U.S. Pat. No. 4,992,244.

Still another approach has been provision of a biosensor in the nature of a sample testing device that includes an electrode structure which makes measurements of one or more electrically measurable characteristics of the sample. The area between two electrodes on one wall of the test cell can be coated with a binding agent which can bind conducting particles such as gold sol particles. See U.S. Pat. No. 5,141,868.

A different type of biosensor which has also been suggested has a thin crystalline drive surfactant polymeric electrically conducting layer to which may be bound members of specific binding pairs. Binding of an analyte or reagent to the binding pair member layer may change electrical properties of the layer for measurement of the analyte. See U.S. Pat. No. 5,156,810.

However, it would still be desirable for the art to have an alternative detection technology which lends itself to ready and versatile implementation as well as consumes power at a very low level, without sacrificing reliability, miniaturization affinity, and low cost.

To those ends, Snow et al. in U.S. Pat. No. 6,221,673 disclosed a method for qualitative and quantitative analysis of chemical species which comprised: (a) exposing to a target environment a device comprising a multiplicity of particles in close-packed orientation, where said particles contained a core of conductive metal or conductive metal alloy and deposited thereon a ligand capable of interacting with chemical species such that a property of said multiplicity of particles is altered; (b) subjecting said multiplicity of particles to conditions sufficient for said property to be exhibited; and (c) monitoring said property to determine any change as an indication of whether, or in what amount, said species is present.

Another chemiresistor approach disclosed in U.S. Pat. No. 6,221,673 made use of a chemically sensitive thin film formed from gold (or possibly other) nanoclusters coated with a monomolecular shell of insulating organic surfactant, e.g., alkanethiols. The extremely small thickness of those shells meant that the intercluster transport was via quantum mechanical tunneling, a phenomenon that is extremely sensitive to dimensional changes associated with vapor sorption. As a result, the response characteristics of that nanocluster sensor were substantially different from earlier chemiresistor approaches. The sensor sensitivity improved from part-per-thousand to sub ppm levels; response time improved from hundreds of seconds to seconds; and conductivity modulation changed from negative only direction to either negative or positive, depending on the chemical nature of the vapor. To reflect those differences, that type of chemiresistor was called a nanocluster MIME (metal-insulator-metal ensemble) sensor.

The nonlinear chemical vapor sensors that are the subject of the present invention are similar in structure to the aforementioned nanocluster MIME sensors of U.S. Pat. No. 6,221,673, however, the overall device size is reduced by a factor of 100 to 1000, from micron to nanometer scales. This dimensional change leads to a highly critical difference in the physics of operation, wherein the nonlinear sensors are believed to operate in a Coulomb blockade regime. Coulomb blockade occurs when the energy required to put an electron on an isolated metal cluster is large when compared to the thermal energy, and when the resistance to electron transfer (usually by tunneling) onto the cluster is also large. If these conditions are satisfied then no current will pass through the island (i.e., it is said to be in “Coulomb blockade”) unless the voltage is large enough to supply the necessary charging energy. As a result, a strongly nonlinear I-V characteristic with a sharp threshold will be observed. Because controlling the tunneling resistance is usually easy (e.g., by increasing the shell thickness of the nanocluster), the key to observing and exploiting Coulomb blockade is the size of the charging energy. For the effect to be strong at room temperature, one must have charging energies much larger than 26 meV (i.e., kT at 300K). The charging energy goes inversely with the size of isolated metal electrode and it turns out that charging energies large enough for room temperature effects are obtained when the size of the metal falls below about 3 nm, a regime easily reached with the nanoclusters employed by the MIME sensor of U.S. Pat. No. 6,221,673.

The primary differences between U.S. Pat. No. 6,221,673 ('673) and the present invention, “Nonlinear Gold Nanocluster Chemical Vapor Sensor”, are summarized as follows:

Operational differences: The chemical sensor of the '673 patent operates in an ohmic regime whereas that of the present invention operates in a strongly nonlinear regime. The nonlinearity of the present invention's sensor provides new capabilities: (a) internal amplification of the chemical signal and thus higher sensitivity; (b) potential for extremely low-power dissipation since the sensor can essentially be “off” when no vapor is present; and (c) potential for digital sensor operation. Note: The ohmic nature of the '673 patent is not stated directly but is implied repeatedly by reference to “the” conductivity of the film.

Structural differences: The nonlinear regime of the present invention's sensor is unexpected and is achieved by a major reduction in device size. This dimensional change is not, however, a simple scaling but involves a set of changes (see table below) all of which are required for the desired operation.

Specific dimensional differences:

| | '673 patent | Current disclosure |
|---|---|---|
| Core size | 0.8 to 40 nm* | <5 nm |
| Shell thickness | 0.4 to 4 nm* | 0.4 to 2 nm |
| Electrode gap | Not stated. 15 μm used in | 10-50 nm; upper limit of |
| | experiments. | 100 nm |
| Electrode and/or | Interdigitated electrodes | <50 nm; upper limit of |
| cluster line width | (which implies very wide). | 100 nm |
| Film thickness | 5 to 10000 nm | <5 nm |

Coulomb blockade effects were first observed in fabricated structures at 4K and have since been seen in many systems and at temperatures up to 300K. Three-terminal operation via electrical gating of the Coulomb blockade has also been widely observed (though only in a few cases at 300K) particularly in the form of single-electron transistor (SET) devices. Such devices can be extremely sensitive, capable of detecting changes in bias charge at levels well below the charge on a single electron. These experiments and devices can be described by a relatively simple model of Coulomb blockade.

The first clear observations of Coulomb blockade in nanoclusters were made using an STM tip to contact a single cluster sitting on a conducting substrate. Later experiments looked at lateral structures with small numbers of clusters positioned between closely spaced electrodes. To simplify the placement of the clusters, only those large in size (5+nm) have been used and the resulting Coulomb blockade signatures, observable only at low temperatures, are less clear than in the STM measurements.

Observing the sharp on-off characteristic and gating effects associated with Coulomb blockade requires structures with very few clusters, however, certain remnants of the Coulomb blockade are still observable even with larger numbers of clusters. For example, nanocluster films consisting of a single (or at most a few) cluster layer(s) deposited between two closely spaced electrodes exhibit a strongly nonlinear current-voltage characteristic (M. G. Ancona et al., Phys. Rev. B, 2001, 64, 033408). This is illustrated in FIG. 1 for a device in which the spacing between electrodes was 39 nm and the transport occurred in a single layer of gold nanoclusters having a core diameter of 1.7 nm. The I-V nonlinearities and the non-zero threshold voltages are most pronounced at cryogenic temperatures but they are also clearly manifested at room temperature. Additional experiments and numerical simulations have shown that these features are consequences of Coulomb blockade in the film. For this regime to be observed the gap between the electrodes must be less than 0.1 micrometer and preferably smaller than 50 nanometer.

### OBJECTS OF THE INVENTION

It is an object of the invention to provide sensitive and reliable technology for the detection and monitoring of chemical species.

It is another object of the invention to provide materials, methods and equipment suitable for the sensitive and reliable detection or quantitation of a preselected chemical species in a target environment.

It is yet another object of the invention to provide materials, methods and equipment as aforesaid which are well-suited for applications requiring compact size, low cost and low power consumption.

It is a further object of the invention to provide the process and methods for fabricating the aforementioned equipment.

### SUMMARY OF THE INVENTION

The invention is a chemiresistor that consists of a very thin film of particles that is deposited on an insulating substrate and is contacted by electrodes. Each of the constituents of this device is described in detail below.

The particles have a metallic core, preferably spheroidal, that is less than 5 nm in diameter and is surrounded by an monolayer ligand shell ranging in thickness from 0.4 nm to 2 nm. The metallic core should be small enough that the electrostatic charging energy of the cluster (i.e., the energy required to put an electron on the cluster) is large compared to the thermal energy (kT˜26 meV at 300K). The ligand shell must be composed of a material that is insulating with an electron barrier height that is also much larger than the thermal energy so that the transport from particle to particle is by quantum mechanical tunneling. Additionally the shell should be thin enough that there is an appreciable probability for electron transfer between particles yet at the same time thick enough that the tunneling resistance is much larger than the resistance quantum (h/4 e²˜6.5 kΩ). Under these conditions the Coulomb blockade effects upon which the subject invention is presumed to rely will be important. One further property of the ligand shell to which there is considerable variability is that its chemical composition can be chosen to be especially receptive to a particular chemical vapor.

The film of particles should be at most a few particles thick and preferably only a single particle layer in thickness. The particles in this layer must form a “close-packed orientation” as illustrated in FIG. 2 in which the ligand shells of neighboring particles are in contact so that electrical conduction can occur from one end of the film to the other. The reason for minimizing the number of particle layers is to reduce the number of conduction paths and thereby strengthen the nonlinearity associated with the Coulomb blockade. When more than a few layers are present, the current-voltage characteristic becomes ohmic and the sensor no longer operates in a nonlinear regime but rather in the same way as the MIME sensor of U.S. Pat. No. 6,221,673.

The electrodes are composed of metal deposited on the substrate. The top surface (at least) of the substrate must be insulating enough to ensure that essentially all of the current between the electrodes flows through the particle film and not through the substrate. The electrodes may be patterned in a variety of geometries but must be spaced no further than 0.1 μm apart; the preferable spacings are in the range 10-50 nm. The electrodes can be defined using optical lithography by first defining widely-spaced electrodes and then doing a second, angled evaporation of metal that narrows the gap down to the size of the “shadow” cast by one of the original electrodes. Gaps in the range of 10-50 nm are easily achieved in this way. Even smaller gaps may be achieved using electroplating techniques. To make a device that not only has a small gap but also a narrow width, one can use standard electron beam lithography. In this case one creates “finger” electrodes with gaps down to 15 nm and widths as small as 25 nm. The reduced width limits the number of conduction paths and thereby strengthens the nonlinearity of the device.

When a voltage is applied across the electrodes of a device configured as described above, a strongly nonlinear current-voltage characteristic with a sharp threshold voltage is measured (when the temperature is such that the previously specified conditions are met). An example of this nonlinear behavior, which is essential to the operation of the invention, is shown in FIG. 1.

The present invention is designed to sense chemical vapors by a transduction of a chemical property change into an electrical signal. When the analyte molecules are adsorbed into the film, they modify the electrical properties of the film and are sensed by a change in the electrical conductivity. This change in conductivity is amplified by the nonlinear and/or threshold behavior of the film I-V characteristic (see FIG. 1) which gives these sensors their high sensitivity.

As noted earlier, the nonlinear chemiresistors of the present invention have much in common with micron-sized MIME sensors of U.S. Pat. No. 6,221,673. In particular, they take advantage of the fact that the particles that serve as the active elements of the micron-sized MIME sensors are extremely small. As a result, the micron-sized devices of the present invention are readily scaled to much smaller dimensions both laterally with more closely-spaced electrodes and vertically with much thinner cluster films including single-layer films. In addition, the small size of the metallic cores of the particles of the present invention means that they have large charging energies and hence can exhibit strong Coulomb blockade effects even at room temperature. Such effects are not observable in the micron-scaled devices because of the huge number of conduction paths that give rise to the overall signal. But as the device is scaled down to a relative few number of particles Coulomb blockade phenomena will come to dominate the behavior and this “new” physics regime can dramatically improve the sensitivity at a given power level of MIME-like chemical vapor sensors. Alternatively, it can achieve the same sensitivity but with much lower power dissipation.

Because the Coulomb-blockade-based chemical vapor sensor described herein passes near zero current when no vapor is present (yet gives a detectable signal upon vapor exposure), it will have ultra-low standby power. This also suggests that the sensor could operate as a digital device and hence require less signal conditioning when interfaced with a digital controller. When vapor is present the signal is strongly amplified by the nonlinear I-V characteristic of the device thus providing high sensitivity at ultra-low power levels. They could function for long periods of time utilizing very weak power sources when operating at pW levels. The sensor can also achieve high selectivity through proper chemical functionalization. Other advantages are: low cost (making possible extensive redundancy); rapid response times; ability to do submicron array sensing; and small thermal mass which provides an extra dimension for vapor detection and discrimination.

### BRIEF DESCRIPTION OF THE DRAWINGS

FIG. 1 is a plot showing the strongly nonlinear current-voltage characteristics of a single-layer film of nanoclusters.

FIG. 2 is a schematic depiction of a basic sensor system in accordance with the invention.

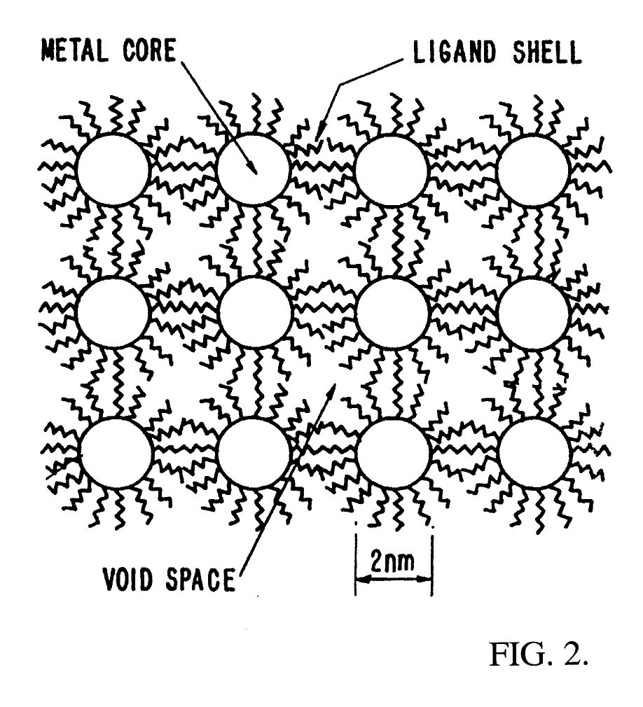

FIG. 3 is a schematic depiction of another sensor system according to the invention, which system includes both a sensor component and a reference component.

FIG. 4 is a schematic diagram of a cluster line sensor.

FIG. 5 is a plot of the current versus time response of the sensor as a result of exposure to piperidine.

### DETAILED DESCRIPTION OF CERTAIN PREFERRED EMBODIMENTS

A central feature of the present invention is a multiplicity of particles in close-packed orientation. Each of the particles is an extremely small cluster of conductive metal atoms that forms a metallic ‘core’ surrounded by a thin ‘ligand shell’ of relatively non-conductive material chemically (e.g., covalently) bound to the core.

The cluster of metal atoms can be composed of a single conductive metal, or of atoms of two or more conductive metals. Suitable conductive metals are metals capable of being processed on a nanoscale and of bonding to a thin insulating ligand shell to form a stabilized metal particle, a multiplicity of which particles is stable in respect of ambient environments and exhibits a stable and measurable electrical conductivity. Examples are noble metals or other conductive metals such as copper, nickel and tin. The elemental metal core is illustratively a noble metal, preferably silver, gold, platinum or palladium. The metal alloy core is illustratively a combination of two or more noble metals, such as two or more of silver, gold, platinum and palladium. The core bodies are advantageously spherical or spheroidal, though they can also be of other regular shapes, or irregular in shape; as will be apparent the shape of the particle typically simulates the shape of the core. Also, typically, the metal cluster core will range from 0.9 to 5 nm (preferably 0.9 to 1.9 nm) in maximum dimension and is preferably spherical. The encapsulating shell has a typical thickness from 0.5 to 2.0 nm, preferably 0.51 to 0.8 nm.

The encapsulating ligand shell is advantageously an organic, inorganic or combined organic/inorganic substance which is preselected for its ability to interact with the chemical species of interest such that the ligand shell is changed in a manner perceptibly affecting a property of the multiplicity of particles, with the result that the species can be detected if present. The ligand molecule typically has a head-tail type structure; the head is a functional group possessing a bonding interaction with metal atoms in the core surface, and the tail has a structure and composition designed to provide additional stabilization of metal clusters (i.e., core bodies) against irreversible agglomeration, induce solubility in solvents and promote interactions with chemical species of interest. The ligand shell can be a monomolecular or multimolecular layer. The ligand shell substance is advantageously a functionalized organic compound, such as a thiol, or an amine. These thiols can be primary aliphatic thiols (preferably straight chain or branched), secondary aliphatic thiols, tertiary aliphatic thiols, aliphatic thiols substituted heterofunctionally (for instance, by OH, COOH, NH₂, Cl, and the like, preferably HS(CH₂)₆OH or the hexafluoroacetone adduct) aromatic thiols, aromatic thiols substituted heterofunctionally (for instance, by OH, COOH, NH₂, Cl, and the like, preferably HS(CH₂)₆OH or the hexafluoroacetone adduct) and araliphatic thiols substituted heterofunctionally (for instance, by OH, COOH, NH₂, Cl, and the like, preferably HS(CH₂)₂OH or the hexafluoroacetone adduct). Preferred amines are primary aliphatic amines. The aliphatic portions of such thiols and amines can be of from 3 to 20 carbon atoms, especially 4 to 16 carbon atoms.

The shell is advantageously neither so thin that the multiplicity of particles is effectively metallic in its conductivity properties, nor so thick that the multiplicity of particles is completely electrically insulating. Preferably, such thickness ranges from 0.4 to 2 nm, especially 0.41 to 0.8 nm. The organic ligand shell stabilizes the metal cluster against irreversible coagulation and also imparts a high solubility of the cluster complex in organic solvents. This allows for processing these materials as thin films.

Once in possession of the teachings herein, one of ordinary in the art will be able to prepare the subject particles by dissolving a salt of the conductive metal—or, in the case of an alloy, salts of the conductive metals—of which the core is to be composed, and an organic substance corresponding to the desired ligand, in a common solvent and subsequently adding a reducing agent under conditions of rapid mixing (see M. Brust et al., J. Chem. Soc., Chem. Comm. 1994, 801; D. V. Leff et al., Langmuir 1996, 12, 4723). The metal ions of the salt(s) are reduced to neutral atoms and subsequently nucleate to form multiatom core bodies. These core bodies grow by absorption of additional metal atoms. Competitively, the organic ligand molecule is absorbed on the growing metal core body surface, encapsulates the metal core body and terminates its growth. The relative concentrations of the metal salts and organic ligand molecules determine the relative rates of metal core body growth and organic ligand encapsulation, and thus the size of the metal core in the stabilized particle. The thickness of the ligand shell is determined by the size of the ligand molecule. It is important that there be a strong chemical interaction between the ligand molecule and neutral metal otherwise the metal core bodies will coagulate and not redisperse. The choice of a suitable ligand molecule is within the skill of the art once the practitioner is in possession of the teaching set forth herein. By way of example, sulfur compounds are particularly effective for coordination to gold, silver, platinum and copper metals. Amines have a weaker but sufficient interaction with gold. In principle, any combination of reducible metal ion and organic ligand, with a sufficient neutral metal to ligand chemical interaction, can form coated metal clusters (i.e., particles) useful in this invention. In other embodiments of the invention alternative synthetic methods can be utilized. For instance, the metal ion reduction can be conducted initially and the deposition of the ligand shell thereafter. This can involve generation of the metal particles in vacuo or in liquid suspension with subsequent formation of the ligand shell by addition of the ligand shell molecules.

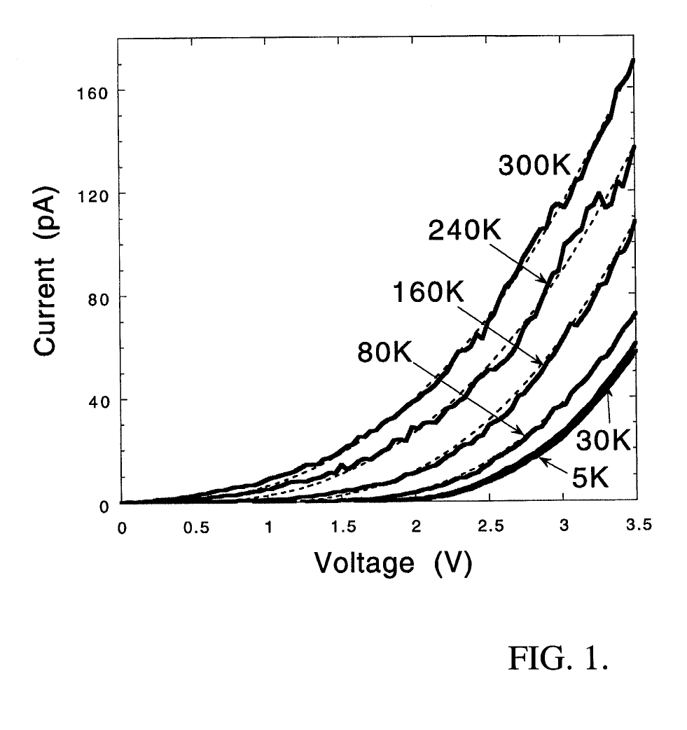

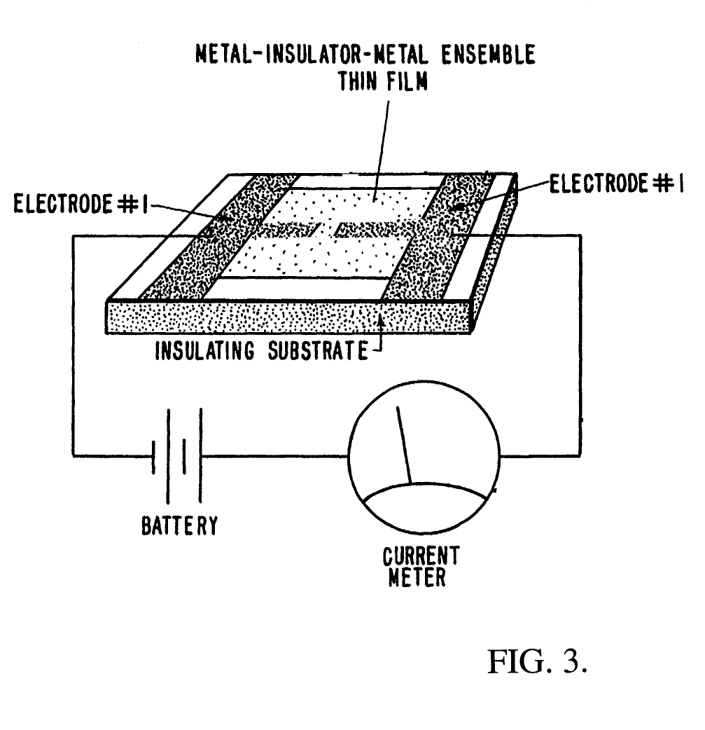

The basic embodiment of a nonlinear chemical vapor sensor is depicted in FIG. 3. This sensor operates in the nonlinear Coulomb blockade regime described above in association with FIG. 1. It consists of a pair of gold electrodes between which is interposed a single-layer film of gold nanoclusters deposited on an insulating substrate. The sensor is fabricated by a two step process in which a pre-patterned substrate is first created and then a directed self-assembly of the nanoclusters is made. The substrate is a silicon wafer capped with a layer of thermal silicon dioxide. The Au electrodes are defined in a “finger” geometry using electron beam lithography. The “finger” electrodes are separated by gaps in the range 5-100 nm and preferred typical widths of 10 to 50 nm.

The self-assembly of the nanoclusters onto the electroded substrates employs two different types of attachment chemistries. In the first clusters are attached to the Au electrodes using α,ω-alkanedithiols as coupling agents. This chemistry can also be used to attach clusters to other clusters in order to form multi-layer films and can even produce finished devices by achieving sufficient thickness to bridge the gap between the electrodes. The second chemical self-assembly technique provides an alternative bridging approach. It attaches clusters directly to SiO₂ surfaces using α,ω-trimethoxy (or trichloro) silylalkanethiol coupling agents. This technique is most useful for producing the single layer films of most interest for this invention. Finally, the significant non-specific adsorption of the gold nanoclusters to SiO₂ can also be used to achieve single-layer films.

After assembly of the nanoclusters onto the substrate a number of chemical modifications can be implemented to boost vapor selectivity and to enhance the Coulomb blockade-mediated effects. Since the latter are primarily influenced by bias charge, polar modifications that induced charge or dipole shifts during vapor sorption/reaction are of most interest.

A further extension of the sensor depicted in FIG. 3 would be to arrange groups of them into a sensor array. Having multiple sensors could provide greatly enhanced selectivity and also redundancy. The selectivity would be achieved by using different cluster coatings in the individual sensors that would have different chemical affinities for the spectrum of possible analytes. Integrating the response information obtained from across the entire array would greatly reduce the possibility of the array being confused, for example, by background vapors such as water.

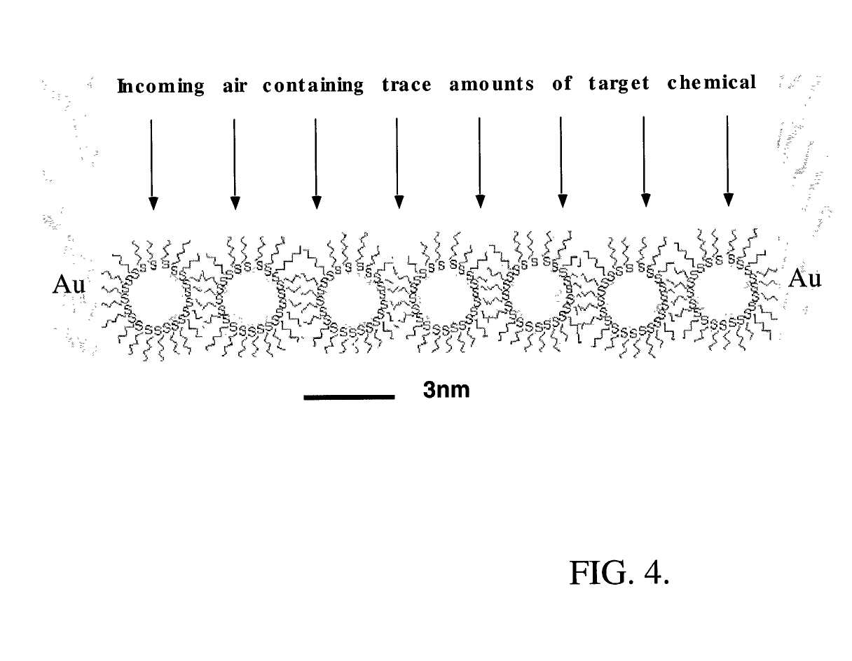

Another extension of the basic embodiment would be to exploit Coulomb blockade more fully to further improve sensitivity. The greatest sensitivity could be achieved if one further reduced the device size so that the sorption of the analyte molecules would modify the Coulomb blockade conditions of a single (or at most a few clusters). In this case, a single electron transistor action would be effected that can be described as a “chemical gating”. It may even be possible for such a sensor to operate at the ultimate limit of single molecule detection. A design for a lateral sensor that would operate in the chemical gating regime is depicted in FIG. 4. (A vertical sensor of this type, e.g., in the STM configuration, would be possible but because of difficulties associated with arranging the sensor housing and the supporting structures and electronics it seems impractical). The fabrication of this sensor is obviously quite difficult as it would require controlling the positions of single nanoclusters that are below 2 nm in size.

### EXAMPLE

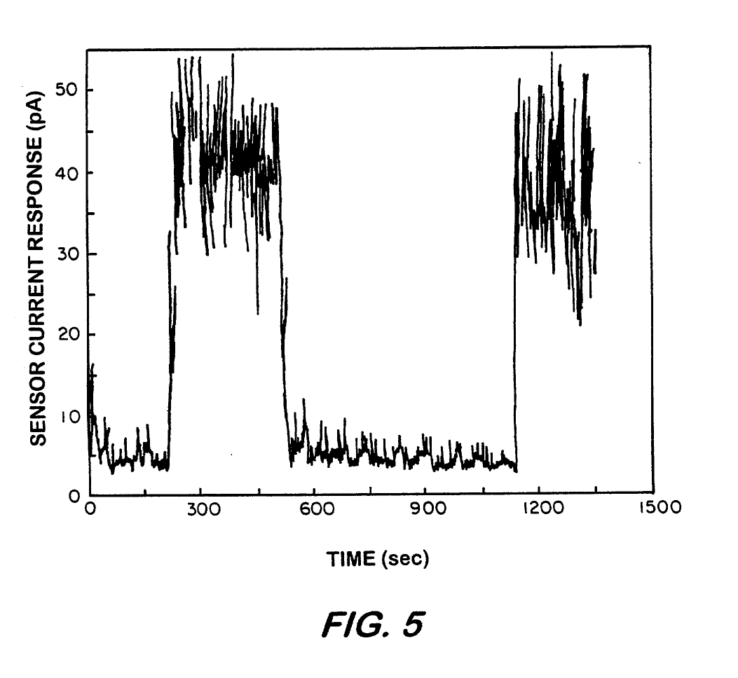

To demonstrate a Coulomb blockade-based nanocluster vapor sensor of the type depicted in FIG. 3, we carried out the fabrication as described above. In order to accentuate the charging effects responsible for Coulomb blockade-mediated detection, following self-assembly of the clusters onto the electronic substrate, an exchange reaction was conducted in which ω-functionalized carboxylic acid-alkanethiols were substituted in place of a fraction of the bound unfunctionalized alkanethiols. The carboxylic acid groups so attached are then available to participate in acid-base interaction with the analyte vapor that would result in charge transfers that would in turn modify the Coulomb blockade conditions in the film thereby affecting conductivity. To illustrate the operation we exposed the sensor to piperidine vapor. At a concentration of 22 parts per thousand the resulting temporal response of the sensor is shown in FIG. 5. The observed increase in current by a factor of 25 is roughly one order of magnitude larger than that seen in a micron-scale device for a similar exposure. This strong amplification occurs because the sensor is operating in the highly nonlinear regime depicted in FIG. 1. It should be noted that the same sensor exposed to a non-reacting vapor like toluene shows barely any response at all. This demonstrates that the sensor is indeed operating in a Coulomb blockade regime with an exquisite sensitivity to charging. And it also shows the potential for high selectivity of such a sensor. One other especially attractive feature of the observed behavior is the near-zero current seen when no vapor is present (FIG. 5). This implies near-zero standby power. Full recovery of the signal is also observed after the sensor housing is purged of piperidine. The response and recovery times are remarkably rapid.

We claim:

1. An article of manufacture, suitable for use in determining whether, or in what amount, a chemical species is present in a target environment, comprising:
an insulating substrate having a top and bottom surface;
two electrodes placed on said top surface of substrate, said electrodes separated by a narrow gap, said gap ranging from 10 to 100 nm across;
a multiplicity of particles in close-packed orientation deposited on said top surface of substrate and within said gap;
said particles having a core of conductive material, said core is from 0.9 to 5 nm in maximum dimension;
said core being encapsulated by a ligand shell, said ligand shell ranging in thickness from 0.4-2.0 nm and which is capable of interacting with said chemical species such that the electron transport of said multiplicity of particles is altered in a detectable manner.

2. An article of manufacture as defined in claim 1, wherein said core conductive material is selected from the group consisting of silver, gold, platinum, palladium, and combinations thereof.

3. An article of manufacture as defined in claim 1, wherein said ligand shell comprises a thiol or an amine.

4. An article of manufacture as defined in claim 3, wherein said ligand shell comprises a thiol selected from the group consisting of primary aliphatic thiols, secondary aliphatic thiols, tertiary aliphatic thiols, heterofunctionally substituted aliphatic thiols, aromatic thiols, heterofunctionally substituted aromatic thiols, and heterofunctionally substituted araliphatic thiols.

5. An article of manufacture as defined in claim 3, wherein said ligand shell comprises an amine selected from the group consisting of primary aliphatic amines.

6. An article of manufacture as defined in claim 1, wherein in said core is from 1.0 to 1.9 nm in maximum dimension and the ligand shell ranges in thickness from 0.41 to 0.8 nm.

7. An article of manufacture as defined in claim 1, wherein said particles are substantially spherical.

8. An article of manufacture as defined in claim 1, wherein said ligand contains a heteroatom functionalized group capable of binding both with the core and the analyte of interest.

9. An article of manufacture as defined in claim 8, wherein said heteroatom is selected from the groups consisting of N, O, S, Cl, F, Br, P, and Si.

10. A method for investigating a target environment to determine whether, or in what amount, a chemical species may be present therein, which comprises:
(a) exposing to said environment an article of manufacture comprising a:
an insulating substrate having a top and bottom surface;
two electrodes placed on said top surface of substrate, said electrodes separated by a narrow gap, said gap ranging from 10 to 100 nm across;
a multiplicity of particles in close-packed orientation deposited on said top surface of substrate and within said gap;
said particles having a core of conductive material, said core is from 0.9 to 5 nm in maximum dimension;
said core being encapsulated by a ligand shell, said ligand shell ranging in thickness from 0.4-2.0 nm and which is capable of interacting with said chemical species such that the electron transport of said multiplicity of particles is altered in a detectable manner;
(b) subjecting said multiplicity of particles to conditions sufficient for said alteration in electron transport to be exhibited; and
(c) monitoring said alteration to determine whether there is, or the amount of, any change as an indication of whether, or in what amount, said chemical species is present.

11. A method as defined in claim 10, wherein said core conductive material is selected from the group consisting of silver, gold, platinum, palladium, and combinations thereof.

12. A method as defined in claim 10, wherein said ligand shell comprises a substance which is capable of interacting with said species such that the conductivity of said multiplicity of particles is altered.

13. A method as defined in claim 10, wherein said ligand shell comprises a thiol or an amine.

14. A method as defined in claim 10, wherein in said core is from 1.0 to 1.9 nm in maximum dimension and the ligand shell ranges in thickness from 0.41 to 0.8 nm.

15. A method as defined in claim 10, wherein the particles are substantially spherical.

16. A method as defined in claim 10, wherein the chemical species, when present, can be detected at an amount of 150 ppm or less.

17. A method for investigating a target environment to determine whether, or in what amount, a chemical species may be present, which comprises:
(a) exposing to said environment an article of manufacture comprising a:
an insulating substrate having a top and bottom surface;
two electrodes placed on said top surface of substrate, said electrodes separated by a narrow gap, said gap ranging from 10 to 100 nm across;
a multiplicity of particles in close-packed orientation deposited on said top surface of substrate and within said gap;
said particles having a core of conductive material, said core is from 0.9 to 5 nm in maximum dimension;
said core being encapsulated by a ligand shell, said ligand shell ranging in thickness from 0.4-2.0 nm and which is capable of interacting with said chemical species such that the electrical conductivity of said multiplicity of particles is altered;
(b) measuring the electrical conductivity of said multiplicity of particles to determine whether there has been, or the amount of, any change in such conductivity compared to the electrical conductivity of such particles not exposed to said environment.

18. A method as defined in claim 17, which comprises exposing the multiplicity of particles to said environment such that, when the chemical species is present, the ligand shell chemically interacts with said species and modulates the current between said particles, with the result that the conductivity of the multiplicity of particles is changed.

19. A method as defined in claim 17, which comprises exposing the multiplicity of particles to said environment such that, when the species is present, the electronic charge distribution of the ligand shell is altered, with the result that the conductivity of the multiplicity of particles is changed.

20. A method as defined in claim 17, which further comprises comparing:
(a) the measurement of the conductivity of said multiplicity of particles exposed to said environment; and
(b) a contemporaneous measurement of the electrical conductivity of a comparable multiplicity of such particles not exposed to said environment.

21. An assembly suitable for investigation of a target environment to determine whether, or in what amount, a chemical species may be present, which comprises:
(a) an article of manufacture comprising a:
an insulating substrate having a top and bottom surface;
two electrodes placed on said top surface of substrate, said electrodes separated by a narrow gap, said gap ranging from 10 to 100 nm across;
a multiplicity of particles in close-packed orientation deposited on said top surface of substrate and within said gap;
said particles having a core of conductive material, said core is from 0.9 to 5 nm in maximum dimension;
said core being encapsulated by a ligand shell, said ligand shell ranging in thickness from 0.4-2.0 nm and which is capable of interacting with said chemical species such that the electron transport of said multiplicity of particles is altered in a detectable manner;
(b) subjecting said multiplicity of particles to conditions sufficient for said alteration in electron transport to be exhibited; and
(c) a sensor for monitoring said alteration in electron transport of said multiplicity of particles.

22. An assembly as defined in claim 21, wherein said core conductive material is selected from the group consisting of silver, gold, platinum, palladium, and combinations thereof.

23. An assembly as defined in claim 21, wherein said ligand shell comprises a substance which is capable of interacting with said species such that the conductivity of said multiplicity of particles is altered.

24. An assembly as defined in claim 21, wherein said multiplicity of particles constitutes a film on said substrate, said film having a thickness of less than 5 nm.

25. An assembly suitable for investigating a target environment to determine whether, or in what amount, a chemical species may be present which comprises:
(a) an article of manufacture comprising a:
an insulating substrate having a top and bottom surface;
two electrodes placed on said top surface of substrate, said electrodes separated by a narrow gap, said gap ranging from 10 to 100 nm across;
a multiplicity of particles in close-packed orientation deposited on said top surface of substrate and within said gap;
said particles having a core of conductive material, said core is from 0.9 to 5 nm in maximum dimension;
said core being encapsulated by a ligand shell, said ligand shell ranging in thickness from 0.4-2.0 nm and which is capable of interacting with said chemical species such that the conductivity of said multiplicity of particles is altered in a detectable manner;
(b) subjecting said multiplicity of particles to conditions sufficient for said alteration in said conductivity to be exhibited; and
(c) a sensor for monitoring the electrical conductivity of said multiplicity of particles to determine whether there is, or the amount of, any change in said conductivity as an indication of whether or in what amount said species is present.

26. An assembly as defined in claim 25, wherein the core comprises gold and the ligand is selected from the group consisting of primary aliphatic thiols, secondary aliphatic thiols, tertiary aliphatic thiols, heterofunctionally substituted aliphatic thiols, aromatic thiols, heterofunctionally substituted aromatic thiols, and heterofunctionally substituted araliphatic thiols.

27. A method of fabricating an assembly suitable for investigation of a target environment to determine whether, or in what amount, a chemical species may be present, which comprises:
(a) depositing on a substrate
(i) a pair of electrodes, said electrodes separated by a narrow gap, said gap ranging from 10 to 100 nm across; said electrodes being electrically connected,
(ii) a thin film of a multiplicity of particles having a core of conductive metal or conductive metal alloy, said core being from 0.9 to 5 nm in maximum dimension, and deposited on said core a ligand shell, of thickness from 0.4 to 2.0 nm, which is capable of interacting with said species such that a property of said multiplicity of particles is altered; and
(b) connecting said pair of electrodes to a sensor capable of determining a change in the property of said multiplicity of particles.

28. A system suitable for investigating a target environment to determine whether, or in what amount, a chemical species may be present, which comprises:
(a) an article of manufacture comprising a:
an insulating substrate having a top and bottom surface;
two electrodes placed on said top surface of substrate, said electrodes separated by a narrow gap, said gap ranging from 10 to 100 nm across;
a multiplicity of particles in close-packed orientation deposited on said top surface of substrate and within said gap;
said particles having a core of conductive material, said core is from 0.9 to 5 nm in maximum dimension;
said core being encapsulated by a ligand shell, said ligand shell ranging in thickness from 0.4-2.0 nm and which is capable of interacting with said chemical species such that a property of said multiplicity of particles is altered;
(d) monitor for monitoring said property to determine whether there is, or the amount of, any change in such property as an indication of whether or in what amount said species is present.

29. A system for investigating a target environment to determine whether, or in what amount, a chemical species may be present, which comprises:
(a) an article of manufacture comprising a:
an insulating substrate having a top and bottom surface;
two electrodes placed on said top surface of substrate, said electrodes separated by a narrow gap, said gap ranging from 10 to 100 nm across;
a multiplicity of particles in close-packed orientation deposited on said top surface of substrate and within said gap;
said particles having a core of conductive material, said core is from 0.9 to 5 nm in maximum dimension;
said core being encapsulated by a ligand shell, said ligand shell ranging in thickness from 0.4-2.0 nm and which is capable of interacting with said chemical species such that electrical resistivity of said multiplicity of particles is altered;
(d) a monitor for monitoring electrical resistivity of said multiplicity of particles to determine whether there is, or the amount of, any change in said resistivity as an indication of whether or in what amount said species is present.

30. A system as defined in claim 30, wherein said monitor used for monitoring the electrical resistivity of said multiplicity of particles includes a current-to-voltage converter circuit followed by a precision rectifier and low-pass filter.

31. A system as defined in claim 31, wherein said monitor further includes a voltage-to-frequency converter.
